SEQUENCE LISTING (2 sequences)

SEQ ID NO: 1 - protein, 17 aa, Streptococcus pyogenes
PDTTYYEEGNEKAEELR

SEQ ID NO: 2 - protein, 15 aa, Streptococcus pyogenes
IKEGDAMWRAPKDRV

(19) United States
(12) Patent Application Publication
(10) Pub. No.: US 2023/0030454 A1
(43) Pub. Date: Feb. 2, 2023

(54) METHOD FOR IDENTIFYING AND TREATING STREPTOCOCCUS PYOGENES INFECTION

(71) Applicant: Chang Gung University, Taoyuan City (TW)

(72) Inventor: Chuan CHIANG-NI, Taoyuan City (TW)

(73) Assignee: Chang Gung University, Taoyuan City (TW)

(21) Appl. No.: 17/385,958

(22) Filed: Jul. 27, 2021

(51) Int. Cl. G01N 33/68; A61K 45/06

(52) U.S. Cl. CPC G01N 33/68; A61K 45/06; G01N 2333/3156; G01N 2333/952; G01N 2800/26

(57) ABSTRACT

Disclosed herein is a method for identifying and treating a Streptococcus pyogenes (group A Streptococcus, GAS) infection in a subject. The method mainly includes determining the presence of endopeptidase O (PepO) protein in the biological sample; and administering an effective amount of an anti-infective agent to the subject to ameliorate symptoms associated with the GAS infection if the PepO protein is present in the biological sample.

### BACKGROUND OF THE INVENTION

### 1. Field of the Invention

The present disclosure relates to disease identification and treatment. More particularly, the disclosure invention relates to a method of identifying and treating a Streptococcus pyogenes (group A Streptococcus, GAS) infection.

### 2. Description of Related Art

Streptococcus pyogenes (group A Streptococcus, GAS) is one of the causative gram-positive bacterial pathogens of cellulitis, necrotizing fasciitis, and toxic shock syndrome, which are collectively known as GAS infection diseases. GAS have more than 250 different emm types, which are classified by the widely used single-locus sequence emm-typing system, and the emm1 and emm3 GAS are considered to be related to severe manifestations. Furthermore, spontaneous mutations in gene encoded for CovR/CovS (control of virulence) two-component regulatory system is also reported to play a role in the bacterial virulence and invasiveness. CovS is the membrane-anchored sensor that modulates the phosphorylation of intracellular CovR with its kinase and phosphatase activities. Spontaneous inactivating mutations in the covR/covS operon resulted in inactivation of CovR phosphorylation, derepression of CovR-controlled virulence factors, and enhancement of bacterial virulence and invasiveness. CovR phosphorylation is also modulated by RocA (regulator of coy) in the CovS-dependent manner. RocA modulates CovR phosphorylation by inhibiting the phosphatase activity of CovS, and the rocA mutant shows the decreased CovR phosphorylation and increased bacterial virulence. Therefore, the inactivating mutations in the covR/covS and the rocA mutant are defined as invasive GAS.

Diseases caused by invasive GAS infection are rapidly progressive and highly destructive, and eventually leading to death. In 2005, there are at least 663,000 new invasive GAS infection cases worldwide and 163,000 deaths each year. For instance, patients of necrotizing fasciitis commonly present with nonspecific symptoms such as fever, vomiting, diarrhea, exquisitely tender skin lesions, and toxemia. The cutaneous manifestations of necrotizing fasciitis are initially absent; hence the correct diagnosis is often mistaken and delayed. As a result, the mortality of necrotizing fasciitis is almost 30% in developed countries, and is over 70% globally. Antibiotics are able to reduce the risk of the invasive GAS infection if administered in early infection; however, occasional antibiotic-resistance occurs and there is no specific and effective biomarker can make early diagnosis for invasive GAS infections.

In view of the foregoing, there exists in the related art a need of a novel method for early identification of invasive GAS infection, so that proper medication may be timely administered to subjects in need of such treatments.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to a method of identifying and treating a subject having a Streptococcus pyogenes (group A Streptococcus, GAS) infection. The method comprises: (a) obtaining a biological sample from the subject; (b) determining the presence of endopeptidase O (PepO) protein in the biological sample; and (c) administering an effective amount of an anti-infective agent to the subject to ameliorate symptoms associated with the GAS infection when the PepO protein is present in the biological sample.

According to some embodiments of the present disclosure, the GAS infection is an invasive GAS infection.

According to some embodiments of the present disclosure, the S. pyogenes has a deletion mutation on covS gene, covR gene, rocA gene, or a combination thereof.

According to alternative embodiments of the present disclosure, the S. pyogenes has a rocA nonsense mutation.

According to some embodiments of the present disclosure, the anti-infective agent is selected from the group consisting of an antibiotic, a vaccine, an antipyretic, and a combination thereof.

Examples of the antibiotic suitable for use in the present disclosure include, but are not limited to, amoxicillin, penicillin, benzathine benzylpenicillin, cefadroxil, cephalexin, clindamycin, azithromycin, clarithromycin, chloramphenicol, erythromycin, spectinomycin, cephalosporins, macrolides, and a combination thereof.

Examples of the antipyretic suitable for use in the present disclosure include, but are not limited to, ibuprofen, naproxen, ketoprofen, nimesulide, salicylate, choline salicylate, magnesium salicylate, sodium salicylate, phenazone, benzocaine, paracetamol, metamizole, and a combination thereof.

In the present disclosure, examples of the biological sample suitable for use herein include, but are not limited to, a whole blood sample, a plasma sample, a serum sample, a urine sample, a saliva sample, a lymph sample, a pleural fluid sample, a mucosa sample, and a combination thereof. According to one working example, the biological sample is a whole blood sample.

Another aspect of the present disclosure is directed to a method of diagnosing a Streptococcus pyogenes (group A Streptococcus, GAS) infection in a subject. The method comprises (a) providing an isolated biological sample from the subject; (b) determining the presence of endopeptidase O (PepO) protein in the isolated biological sample; and (c) making a diagnosis based on the presence or absence of the PepO protein in the isolated biological sample.

According to some embodiments of the present disclosure, the GAS infection is an invasive GAS infection.

According to some embodiments of the present disclosure, the S. pyogenes has a deletion mutation on covS gene, covR gene, rocA gene, or a combination thereof.

According to other embodiments of the present disclosure, the S. pyogenes has a rocA nonsense mutation.

Examples of the isolated biological sample suitable for use in the present disclosure include, but are not limited to, a whole blood sample, a plasma sample, a serum sample, a urine sample, a saliva sample, a lymph sample, a pleural fluid sample, a mucosa sample, and a combination thereof. According to one working example, the biological sample is a whole blood sample.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a photograph depicting the results of identification of an unknown protein marker for CovR/CovS-inactivated isolates. CovR/CovS-inactivated mutations (i.e., covS mutant [ΔcovS]) result the inactivation of CovR phosphorylation. The upper panel shows the absence of phosphorylated CovR protein (CovR˜P) in different emm-type clinical isolates detecting by Phos-tag western blot hybridization. Wide type (as “Wt” indicated in figure) is the emm1-type wild-type strain (A20) and utilizes as the positive control. The lower panel shows the total protein profile on SDS-PAGE. Arrow indicates the specific protein signal was only found in clinical isolates that did not express phosphorylated CovR protein.

FIG. 2 is a photograph depicting the results of assessment of total PepO level in covS mutant strains. The total protein profile of the wild-type strain (Wt), covS mutant strain (ΔcovS), covS and pepO double-mutant strain (ΔcovSApepO), and pepO trans-complementary strain (ΔcovSApepO+PpepO) are presented on SDS-PAGE gel. Arrow indicates the protein marker for covS mutant. This protein marker was absent in the covS and pepO double-mutant strain but restored in the pepO trans-complementary strain, suggesting PepO is the specific marker for covS mutant.

FIG. 3 is a photograph depicting the result of verifying whether the PepO protein is present in CovR/CovS-inactivated mutants. The upper panel shows the result of Phos-tag western blot hybridization, “CovR˜P”, phosphorylated CovR; the middle panel shows the total protein profile (SDS-PAGE); and the lower panel depicts the expression of PepO (western blot hybridization) in the wild-type strain (Wt), covR deletion mutant strain (ΔcovR), covS deletion mutant strain (ΔcovS), and selected clinical isolates, respectively. Arrow indicates the specific protein signal for CovR/CovS-inactivated mutants.

FIG. 4 is a photograph depicting the results of verifying whether the PepO protein is present in various emm-type isolates. Different clinical emm-type isolates with or without phosphorylated CovR were included for western blot hybridization using anti-PepO antibodies. CovR˜P: phosphorylated CovR protein. Arrow indicates the specific protein signal for CovR/CovS-inactivated mutants.

FIG. 5 is a photograph depicting the results that PepO as the marker for the identification of rocA mutants and emm3-type clinical isolates. The rocA gene of emm3-type isolates is truncated. The upper panel shows the result of Phos-tag western blot hybridization of the phosphorylation level of CovR (CovR˜P in FIG. 5); the middle panel reveals the result of western blot hybridization showing that the PepO expresses in mutant strains (ΔrocA, rocAₑₘₘ₃ and selected emm3 clinical isolates). The lower panel shows the result of total protein profile (SDS-PAGE) of the emm1-type wild-type strain (Wt), rocA-deletion mutant (ΔrocA), rocA truncated mutant (rocAₑₘₘ₃), and selected emm3 clinical isolates, respectively. Arrow indicates the specific protein signal for CovR/CovS-inactivated mutants.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. DEFINITIONS

For convenience, certain terms employed in the specification, examples and appended claims are collected here. Unless defined otherwise, all technical and scientific terms used herein have the same meaning as commonly understood by one of the ordinary skill in the art to which this invention belongs.

The singular forms “a”, “and”, and “the” are used herein to include plural referents unless the context clearly dictates otherwise.

The term “invasive group A Streptococcus (GAS) infection” refers to a severe and sometimes life-threatening infection, in which the bacteria (e.g., Streptococcus genus including S. pyogenes) have invaded parts of the body, such as the blood, deep muscle and fat tissue, and the lungs, thereby leads to severe illness of the host. Invasive GAS infections are more aggressive than common non-invasive GAS infections that includes strep throat, scarlet fever, impetigo, and ear infections, and often cause conditions like streptococcal toxic shock syndrome and necrotizing fasciitis.

The term “biological sample” refers to any sample including fluid samples (such as body fluids samples and urine samples) and tissue samples (such as tissue sections and needle biopsies of a tissue); cell samples (e.g., cytological smears (such as Pap or blood smears) or samples of cells obtained by microdissection); or cell fractions, fragments, organelles (such as obtained by lysing cells and separating the components thereof by centrifugation or otherwise) or microorganisms (such as obtained by culturing the said samples in a proper environment). Other examples of biological samples include whole blood, serum, plasma, urine, saliva, lymph, mucosa, pleural fluid, or a combination thereof.

An “effective amount” of an anti-infective agent described herein refers to an anti-infective agent in an amount effective, at dosages, and for periods of time necessary, to elicit the desired biological response, i.e., treating the infection. As will be appreciated by those of ordinary skill in this art, the effective amount of the anti-infective agent described herein may vary depending on such factors as the desired biological endpoint, the pharmacokinetics of the agent, the condition being treated, the mode of administration, and the age and health of the subject. An effective amount of the anti-infective agent described herein means an amount of anti-infective agent, alone or in combination with other therapeutic means, which provides a therapeutic benefit in the treatment of the infection. In certain embodiments, an effective amount is a therapeutically effective amount. In certain embodiments, an effective amount is a prophylactically effective amount. According to some embodiments of the present application, the amount of a compound described herein in a single dose. In certain embodiments, an effective amount is the combined amounts of a compound described herein in multiple doses.

A “therapeutically effective amount” of an anti-infective agent described herein is an amount sufficient to provide a therapeutic benefit in the treatment of an infection or to ameliorate or minimize one or more symptoms associated with the infection, so as to achieve the desired therapeutically desired result with respect to the treatment of GAS infections caused by Streptococcus pyogenes. A therapeutically effective amount of an anti-infective agent means an amount of anti-infective agent, i.e., an antibiotic, alone or in combination with other therapeutic means, which provides a therapeutic benefit in the treatment of the GAS infection. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount sufficient to prevent an infection, or one or more symptoms associated with the infection or prevent its recurrence. A prophylactically effective amount of a compound means an amount of a therapeutic agent, i.e., a vaccine, alone or in combination with other agents, which provides a prophylactic benefit in the prevention of the infection, or at least preventing the severe symptom associated with the infection. The term “prophylactically effective amount” can encompass an amount that improves overall prophylaxis or enhances the prophylactic efficacy of another prophylactic agent.

The term “anti-infective” agent used in the present disclosure refers to a compound or a composition that has therapeutic benefits against the infectious diseases. Particularly in the present application, the anti-infective agent is able to prevent or inhibit the growth of pathogens (i.e., S. pyogenes in the present disclosure), or to ameliorate the symptoms caused by GAS infection, such as, fever, pain and/or toxemia. According to the present disclosure, the anti-infective agent may be an antibiotic, a vaccine, an antipyretic, or a combination thereof.

The term “subject” or “patient” is used interchangeably herein and is intended to mean a mammal including the human species that is treatable by the method of the present disclosure. The term “mammal” refers to all members of the class Mammalia, including humans, primates (e.g., monkey, and chimpanzee), domestic and farm animals, such as rabbit, pig, goat, sheep, and cattle; as well as zoo, sports or pet animals (e.g., a horse, a dog, a cat and etc); and rodents, such as mouse, rat, guinea pig, and hamster. In a working example, the subject is a human. Further, the term “subject” or “patient” intended to refer to both the male and female gender unless one gender is specifically indicated.

A “deletion mutation” means a type of mutation that involves the loss of genetic material, including deoxyribonucleotide, nucleotide, DNA, gene or chromosome which may be from a single base to an entire piece of chromosome. Deletion of one or more nucleotides in the DNA leads to an altered reading frame or non-reading frame of the gene; hence, it could result in a complete failure on the synthesis of the gene product, or cause a nonfunctional gene product (e.g, a loss-of-function mutation). In some embodiments of the present disclosure, one or more deletion mutations occur on covS, covR, or rocA genes, causing the inactivated protein function (e.g., an inactivated phosphorylation).

### 2. DETAIL DESCRIPTION OF PREFERRED EMBODIMENTS

The present disclosure is based, at least in part, on the discovery that endopeptidase O (PepO) protein is overexpressed in invasive S. pyogenes isolates but not in non-invasive S. pyogenes isolates. Hence, the increased PepO expression may be used as an indicator for early identification of GAS infection in a subject, so that proper medication is timely administered to ameliorate symptoms associated therewith GAS infection.

### 2.1 Identification and Treatment of a Subject Having a GAS Infection

The first aspect of the present disclosure aims at providing a method of identifying and treating a subject having a GAS infection, more particularly, the invasive GAS infection.

One aspect of the present disclosure is directed to a method of identifying and treating a subject having a GAS infection. The method comprises:

(a) obtaining a biological sample from the subject;

(b) determining the presence of endopeptidase O (PepO) protein in the biological sample; and

(c) administering an effective amount of an anti-infective agent to the subject to ameliorate symptoms associated with the GAS infection when the PepO protein is present in the biological sample.

The present method consists of two parts, that is, identification steps and treatment steps. For the purpose of identification, a biological sample is first obtained from the subject. Examples of biological samples suitable for use in the present method include, but are not limited to, a whole blood sample, a serum sample, a plasma sample, a urine sample, a saliva sample, a pleural fluid sample, a lymph sample, a mucosa sample, and a combination thereof. In one working example, the biological sample is a whole blood sample obtained from a subject suspected of having a GAS infection. Optionally, the biological sample thus obtained may be subjected to a bacterial culture by any means known to those skilled persons in the art. Typical methods and materials for incubating S. pyogenes are described, for example, in Gera & Mclver, “Laboratory Growth and Maintenance of Streptococcus pyogenes (The Group A Streptococcus, GAS)” Curr Protoc Microbiol. 30: 9D.2.1-9D.2.13 (2013); which is incorporated herein by reference. In general, biological samples (e.g., a fluid sample or a whole blood sample) are collected and cultivated in suitable conditions for a period of time until the bacterial colonies are formed. In one working example, the S. pyogenes strains are cultivated in a trypticase soy agar supplemented with yeast extraction. In another working example, the S. pyogenes strains are cultivated in tryptic soy broth supplemented with yeast extraction. After incubation, the bacterial colonies are collected and lysed until further used.

The bacteria lysate described above is then subjected to analysis of the presence of endopeptidase O (PepO) protein (step (b)). The presence of PepO protein in the lysate may be determined by any means known in the art, which include but are not limited to, gel electrophoresis (e.g., western blot, Phos-tag SDS-PAGE), isotope labeling (e.g., isotope-coded affinity tag (ICAT)), mass spectrometry (MS) (e.g., LC-MS, MALDI-TOF, etc.), enzyme-linked immunosorbent assay (ELISA), immunohistochemistry (IHC), and etc. In some embodiments, the presence of PepO protein is determined by western blot. In other embodiments, the presence of overexpressed PepO protein is determined by Phos-tag SDS-PAGE hybridization. In further embodiments, the presence of overexpressed PepO protein is determined by ELISA. According to preferred embodiments of the present disclosure, the presence of overexpressed PepO protein indicates that the subject has a GAS infection. In some preferred embodiments, the overexpression of PepO protein refers to an increased expression of PepO protein in the bacterial lysate (i.e., S. pyogenes lysate) as compared to that of a control. Preferably, the S. pyogenes are those having any of covS, covR, and rocA mutants or a combination thereof instead of wild-type, non-invasive strain of S. pyogenes. In such case, the subject is determined to be one that suffers from a GAS infection, specifically, an invasive GAS infection. In working examples, the presence of overexpressed PepO protein is determined via hybridization with specific anti-PepO antibodies. Specifically, antibodies specific to the antigenic epitope of PepO protein are suitable for used in the present method. The epitope sequence of PepO can be predicted and determined through any published sequence database by any means known to those skilled persons in the art. In some examples, the epitope sequences of PepO protein recognized by specific anti-PepO antibodies may be “PDTTYYEEGNEKAEELR” (SEQ ID No: 1) or “IKEGDAMWRAPKDRV” (SEQ ID No: 2). Antibody specific to any of the epitope sequences of PepO protein may be used to determine the presence of PepO protein in the bacterial lysis derived from or in the biological sample.

Once the subject is diagnosed to be infected by S. pyogenes, then an effective amount of an anti-infective agent may be administered to the subject so as to ameliorate symptoms associated with the GAS infection.

According to some embodiments of the present disclosure, the subject has an invasive GAS infection, in which the S. pyogenes has a deletion mutation on covS gene, covR gene, rocA gene, or a combination thereof. Optionally or alternatively, the subject has an invasive GAS infection, in which the S. pyogenes has a rocA nonsense mutation. Any of these mutations will result in inactivation of CovR phosphorylation and enhancement of bacterial invasiveness.

Examples of anti-infective agents suitable for use in the present method (i.e., for administering to a subject, whose biological samples or fluids biopsy are determined to have overexpressed PepO protein of S. pyogenes) include, but are not limited to, an antibiotic, a vaccine, an antipyretic, and a combination thereof. Examples of antibiotics suitable for use in the present method includes, but are not limited to, amoxicillin, penicillin, benzathine benzylpenicillin, cephalosporin and its derivatives (e.g., cefadroxil, cephalexin, cefaloglycin, cefalonium . . . etc), clindamycin, macrolide and its derivatives (e.g., azithromycin, clarithromycin, erythromycin, fidaxomicin . . . etc), chloramphenicol, erythromycin, spectinomycin, and a combination thereof. Examples of vaccines suitable for use in the present method include a vaccine that specific to group A Streptococcus. Examples of antipyretics suitable for use in the present method include, but are not limited to, ibuprofen, naproxen, ketoprofen, nimesulide, salicylate, choline salicylate, magnesium salicylate, sodium salicylate, phenazone, benzocaine, paracetamol, metamizole, and a combination thereof. Any clinical artisans may choose a suitable agent for use in the present method based on factors such as the particular condition being treated, the severity of the condition, the individual patient parameters (including age, physical condition, size, gender and weight), the duration of the treatment, the nature of concurrent therapy (if any), the specific route of administration and like factors within the knowledge and expertise of the health practitioner.

### 2.2 In Vitro Diagnosis of GAS Infection

Also disclosed herein is a method of diagnosing a S. pyogenes (GAS) infection in a subject via use of an isolated biological sample. The method comprises:

(a) providing an isolated biological sample of the subject;

(b) determining the presence of endopeptidase O (PepO) protein in the isolated biological sample; and

(c) making a diagnosis based on the presence of the PepO protein in the isolated biological sample determined in the step (b).

As described in Section 2.1 of this paper, a biological sample (e.g., a urine sample, a blood sample and etc) is first isolated from the subject, and preferably, the isolated biological sample is subjected to further cultivation for S. pyogenes. To make a diagnosis on whether the subject has a GAS infection, the expression of PepO protein in the isolated biological sample (i.e., the cultivated bacterial lysate) is determined via any means known in the related art (e.g., ELISA, western blot and etc). Then, the level of PepO protein in the biological sample is compared with that of a control sample, which is obtained from a healthy subject or a non-S. pyogenes infectious subject, noted that non-S. pyogenes pathogen does not express PepO. Accordingly, if PepO protein is detected in the biological sample, it means the biological sample indeed contains S. pyogenes, thus the subject has a GAS infection. In some embodiments, the S. pyogenes strain that expresses PepO protein has a deletion mutation on covS gene, covR gene, rocA gene, or a combination thereof. Additionally or alternatively, the S. pyogenes strain that expresses PepO protein has a rocA nonsense mutation. The mutant strains of S. pyogenes are known to cause invasive GAS infection.

By the virtue of the above features, the present method can provide early identification and detection of S. pyogenes infection, particularly the group of S. pyogenes unable to be identified by conventional colony morphology test. In addition, the present method can increase the sensitivity of the invasive group A Streptococcus infection, thereby allowing the identified patients to be treated in time and properly.

### EXAMPLES

### Materials and Methods

### Bacterial Strains and Culture Conditions

S. pyogenes A20 and AP3 strains and various emm-types of clinical isolates were used in this study. S. pyogenes strain A20 is the well-reported emm1-type strain which was described in previous studies (Chiang-Ni et al., “emm1/sequence type 28 strains of group A Streptococci that express covR at early stationary phase are associated with increased growth and earlier SpeB secretion”, J Clin Microbiol 47:3161-3169, 2009). S. pyogenes strain AP3 is an animal passage isolate of A20 that has a frameshift deletion in the covS gene; and the covR gene was interrupted by a chloramphenicol (Cm) cassette in A20 strains to construct an inactivating covR mutant (Chiang-Ni et al., “Repression of Rgg but not upregulation of LacD.1 in emm1-type covS mutant mediates the SpeB repression in group A Streptococcus” Front Microbiol 7:193 5, 2016). The deletion or truncated mutation in the rocA gene were constructed from A20 strain with the method described by Chiang-Ni et al. (“RocA regulates phosphatase activity of virulence sensor CovS of group A Streptococcus in growth phase- and pH-dependent manners.” mSphere 5:3 2020.) Clinical isolates that numbered according to emm-types were described and analyzed in our previous study (Chiang-Ni et al., “Epidemiology analysis of Streptococcus pyogenes in a hospital in Southern Taiwan by use of the updated emm cluster typing system.” J Clin Microbiol 54:157-162, 2016). In addition to emm1, further 15 emm-types, which are emm3, emm12, emm22, emm25, emm41, emm49, emm73, emm78, emm81, emm87, emm89, emm90, emm92, emm102, and emm113, were utilized in this study. All strains were cultured on trypticase soy agar containing 5% sheep blood or in tryptic soy broth (Becton Dickinson and Company) supplemented with 0.5% yeast extract (hereinafter, TSBY broth). E. coli DH5α purchased from commercial supplier (Yeastern Biotech Co., LTD) was used as the control to test the transformation efficiency and was cultured in lysogeny broth (LB) at 37° C. with vigorous aeration. The antibiotics used for selection were chloramphenicol (25 μg/mL and 3 μg/mL for E. coli and S. pyogenes strains, respectively), erythromycin (125 μg/mL for E. coli and 5 μg/mL for S. pyogenes strains, respectively), and spectinomycin (100 μg/mL for both E. coli and S. pyogenes strains).

### Construction of pepO Mutant and pepO Trans-Complementary Strains

Bacterial genomic DNA extraction was performed in accordance with method described by Wang et al (“Peroxide Responsive Regulator PerR of group A Streptococcus Is Required for the Expression of Phage-Associated DNase Sda1 under Oxidative Stress”, PLoS One 8:e81882, 2013). To construct the pepO isogenic mutant, plasmid pCN210 was transformed into AP3 strain via electroporation, and the transformants were selected as according to the protocol described by Chiang-Ni et al., (Front Microbiol 7:1935, 2016). The deletion of pepO gene in the transformants was confirmed via Sanger sequencing. To perform the complementation expression on the deleted pepO gene, the low copy number vector pTRKL2 carried the pepO gene (NCBI accession number: M5005_Spy1782; 1896 bp) with its native promoter (upstream 493 bp) was transformed into the pepO mutant construct. The transformants were selected by erythromycin.

### Anti-PepO Antibodies

Two short peptides, PDTTYYEEGNEKAEELR (SEQ ID No: 1) and IKEGDAMWRAPKDRV (SEQ ID No: 2), respectively directed to antigenic regions of PepO protein, were synthesized and utilized to immunize rabbits for antibody production (Leadgene Biomedical, Inc.). The anit-PepO antibodies thus obtained were termed Ab-PDTT and Ab-IKEG.

### Western Blot and Phos-Tag Western Blot

Bacteria were cultured in TSBY broth to the turbidity of O.D.₆₀₀ 1.0, then the bacterial colonies were collected and disrupted by using a bead beater (Mini-Beadbeater, BioSpec Products Inc.). The bacterial cell lysate was centrifuged, and total protein in supernatants were collected for further analysis. Total protein of the lysate was mixed with 6× protein loading dye, boiled for 5 min, and loaded onto 10% SDS-PAGE gel.

For Phos-tag western blot analysis, the bacterial total proteins were mixed with 6× protein loading dye (without boiling) and loaded into a 10% SDS-PAGE gel containing 10 μM of Phos-tag (Wako Pure Chemical Industries Ltd.) and 0.5 μM MnCl₂. The separated proteins were transferred onto polyvinylidene fluoride membranes (Millipore; Billerica). The membranes were blocked with 5% skim milk in PBST buffer (PBS containing 0.2% v/v Tween-20) at 37° C. for 1 h. CovR protein was detected using anti-CovR serum (Chiang-Ni et al., Front Microbiol 7:1935, 2016), SLO protein was detected by anti-SLO antibody (Genetex, Irvine, Calif., USA), and PepO was detected by antibodies Ab-PDTT or Ab-IKEG. After hybridization, the membrane was washed with PBST buffer and hybridized with a secondary antibody, peroxidase-conjugated goat anti-rabbit IgG (Cell Signaling Technology, Inc.) at room temperature (25° C.-28° C.) for 1 h. The blot was developed using a special western blotting substrate (Pierce ECL Substrate, Thermo Fisher Scientific Inc.) and the signals were detected using an image system (Gel Doc XR+system, Bio-Rad Laboratories, Inc.).

### Example 1 Identification of PepO Protein in CovR and CovS Mutant Strains

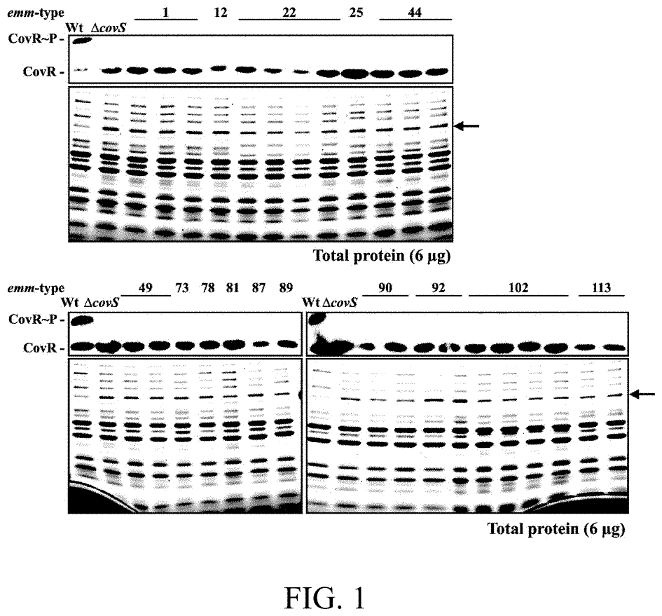

In this example, the differences of protein expression profile among various S. pyogenes strains were investigated, and results are provided in FIG. 1.

It was found that an unknown protein (˜70 kDa) was over-expressed in many covR/covS inactivated mutant strains of S. pyogenes but not in wild type strain. Specifically, this unknown protein was only clearly visualized on 10% SDS-PAGE gel in CovS kinase-inactivated mutant (i.e., ΔcovS) and emm-type clinical isolates (no: 1, 12, 22, 25, 44, 49, 73, 78, 81, 87, 89, 90, 92, 102 and 113), but not in the functional phosphorylated CovR strain (wild type), which indicated that inactivation of CovS/CovR triggered the over-expression of this unknown protein. By mass spectrometry analysis, three candidates of this unknown protein were identified, including endopeptidase PepO (72 kDa), proline tRNA ligase (69 kDa), and oligoendopeptidase F (69 kDa). Based on a previous study of Wilkening et al. (Mol Microbiol 99:71-87, 2016), in which they taught that the transcription of pepO gene was 2.5-4-fold upregulated in the covR mutant (ΔcovR) and the phosphorylation dysfunction strain, compared to that in the wild-type strain, we thus hypothesized that the unknown protein may be PepO protein.

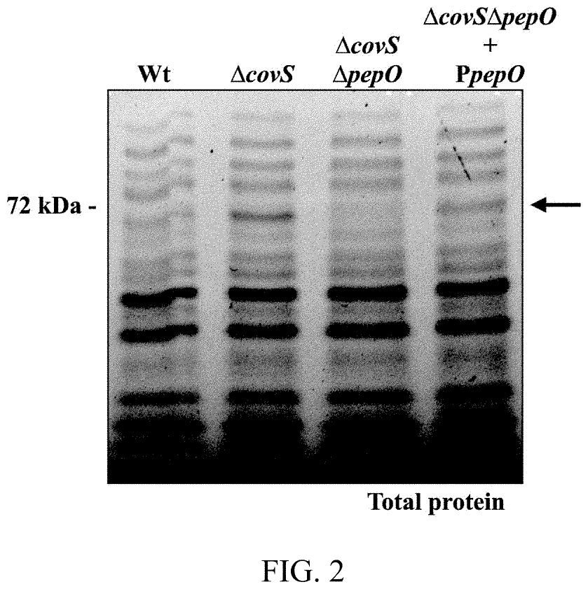

To verify whether this unknown protein was indeed PepO, the covS and pepO double mutant strain was constructed and total proteins of the wild-type strain, covR mutant, covS mutant, and covS and pepO double mutant strains were individually extracted and analyzed by SDS-PAGE. Results are shown in FIG. 2.

It was found that the 70 kDa protein signals were absent in the wild-type strain, and the covS and pepO double mutant strain. Furthermore, the expression of this 70 kDa protein was restored in the pepO trans-complementary strain (see the far-right lane of FIG. 2), confirming this 70 kDa protein was indeed PepO.

### Example 2 PepO Protein Plays a Role in CovR/CovS-Inactivated Mutant Strains

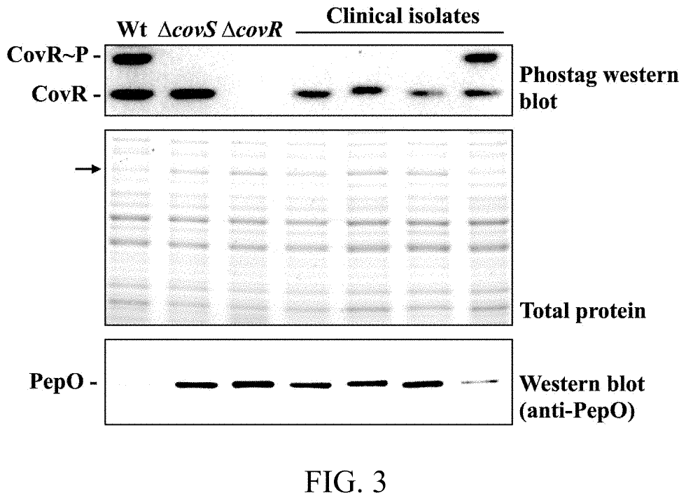

In this experiment, whether PepO could serve as a marker for identification of group A Streptococcus (GAS) infection was investigated, To this purpose, the presence of PepO in bacterial isolates (e.g., those with CovR/CovS-inactivated mutations) was determined by western blot, and the two polyclonal antibodies, Ab-PDTT (0.1 μg/mL) and Ab-IKEG (0.1 μg/mL), were used to recognize PepO protein. The covS/pepO double mutant strain was used for negative control. Results are depicted in FIG. 3.

It was found that both covS mutant and covR mutant individually showed increased PepO expression, as compared to that in the wild-type strain. On the other hand, no signal for size around 70 kDa was detected in negative control (i.e., covS/pepO double mutant; data not shown). In line with the total protein SDS-PAGE analysis results (See middle panel of FIG. 3), western blot analysis also suggested PepO could serve as the specific marker for identifying covR/covS mutants (see lower panel of FIG. 3).

### Example 3 PepO Protein is a Marker for Identifying Clinical emm-Type Isolates

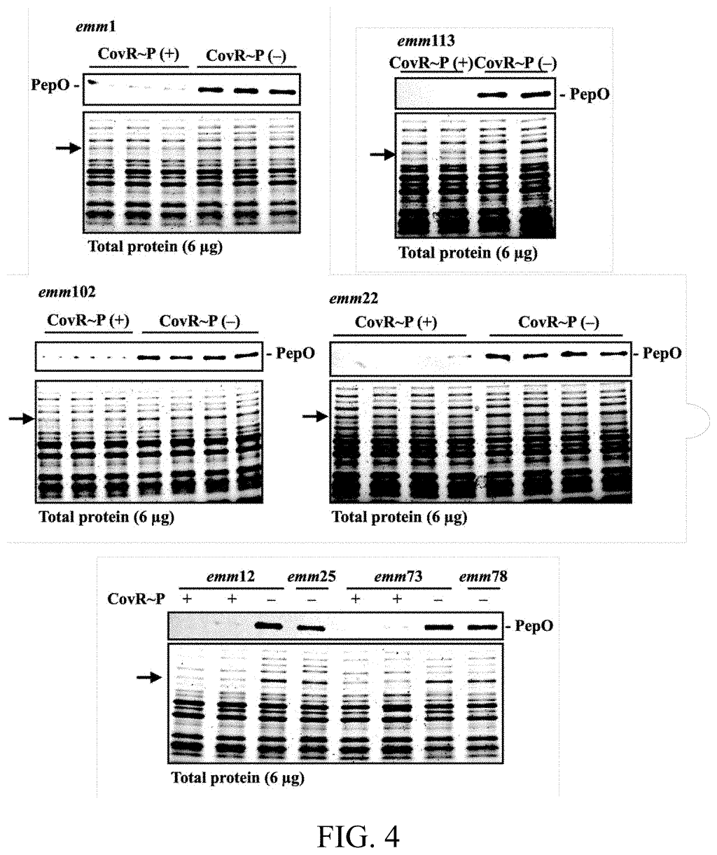

In this experiment, the specificity of PepO protein as a biomarker for invasive emm-type isolates was further investigated. Eight clinical isolates, i.e., emm1, emm12, emm22, emm25, emm73, emm78, emm102, and emm113, were used in this experiment; and western blot and anti-PepO antibody (Ab-PDTT, 0.1 μg/mL) were employed to investigate whether PepO may identify the CovR/CovS-inactivated mutation in those clinical isolates. Each emm-type isolates comprised either CovR/CovS-inactivated mutation that lacked phosphorylation of CovR protein or a normal functional CovR/CovS; hence, by comparing the PepO level in these CovR/CovS-inactivated isolates with that of the same emm-type isolates of normal functional CovR/CovS would confirm whether PepO may act as the indicator for identifying invasive emm-type isolates. Results are depicted in FIG. 4.

As the western blot shown in FIG. 4, in the same isolate, the one that had CovR/CovS-inactivated mutation exhibited higher PepO level than that of the isolate with a normal functional CovR/CovS system, and such expression profile was consistent among different clinical isolates.

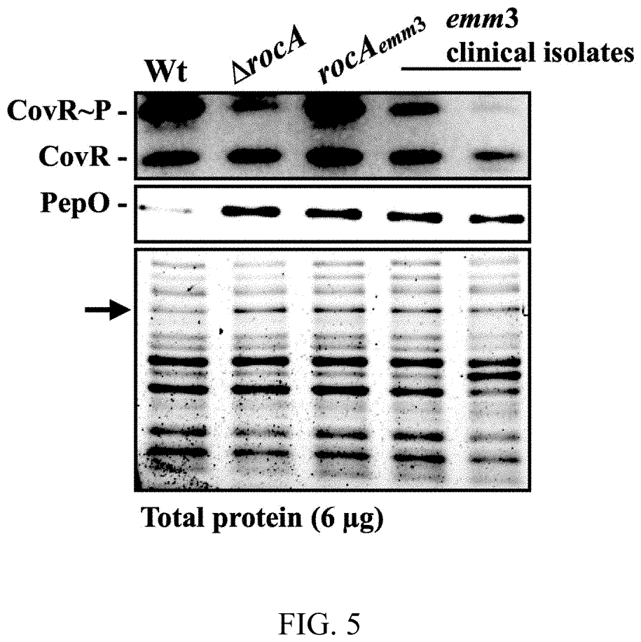

Further, it has been reported that the upstream regulator of CovR/CovS, rocA gene, is associated with CovR/CovS system, and the mutations in rocA gene inactivate CovR phosphorylation. Since the emm3 -type isolates have the truncated rocA gene and express lower level of phosphorylated CovR than that in wild type strains (e.g., emm1-type isolate or A20 strain), the specific emm3 isolate is ideal for verifying the genetic regulation for the expression of PepO protein. In this study, whether PepO could identify the rocA mutant in emm isolates was verified. The A20 (emm1) rocA isogenic mutant, A20 with truncated rocA gene, and two clinical emm3 isolates were utilized for this analysis. Results are provided in FIG. 5.

As depicted in FIG. 5, the rocA mutants and emm3 clinical isolates expressed detectable level of phosphorylated CovR, and the expression of PepO was significantly upregulated in the rocA mutants and emm3 isolates compared to wild-type A20 strain. Furthermore, the PepO signal can be clearly detected by both SDS-PAGE gel and western blot hybridization in the rocA mutants and emm3 isolates as depicted in the middle panel of FIG. 5, indicating that PepO protein is a potential marker for detecting rocA mutants and emm3 isolates.

Taken together, the increase of PepO expression was detected in both covR and covS mutants, further suggesting PepO would be a reliable marker of identification of CovR/CovS-inactivated isolates. In addition, the expression of PepO was upregulated in the rocA mutant and emm3 isolate compared to that in the wild-type strain, indicating the overexpression of PepO protein is capable of being a specific marker for identifying RocA-inactivated isolates, including emm3-type isolate.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for identifying and treating a subject having a Streptococcus pyogenes (group A Streptococcus, GAS) infection, comprising:
(a) obtaining a biological sample from the subject;
(b) determining the presence of endopeptidase O (PepO) protein in the biological sample; and
(c) administering an effective amount of an anti-infective agent to the subject to ameliorate symptoms associated with the GAS infection when the PepO protein is present in the biological sample.

2. The method of claim 1, wherein the GAS infection is an invasive GAS infection.

3. The method of claim 1, wherein the S. pyogenes has a deletion mutation on covS gene, covR gene, rocA gene, or a combination thereof.

4. The method of claim 1, wherein the S. pyogenes has a rocA nonsense mutation.

5. The method of claim 1, wherein the anti-infective agent is selected from the group consisting of an antibiotic, a vaccine, an antipyretic, and a combination thereof.

6. The method of claim 5, wherein the antibiotic is selected from the group consisting of amoxicillin, penicillin, benzathine benzylpenicillin, cefadroxil, cephalexin, clindamycin, azithromycin, clarithromycin, chloramphenicol, erythromycin, spectinomycin, cephalosporins, macrolides, and a combination thereof.

7. The method of claim 5, wherein the antipyretic is selected from the group consisting of ibuprofen, naproxen, ketoprofen, nimesulide, salicylate, choline salicylate, magnesium salicylate, sodium salicylate, phenazone, benzocaine, paracetamol, metamizole, and a combination thereof.

8. The method of claim 1, wherein the biological sample is selected from group consisting of a whole blood sample, a plasma sample, a serum sample, a urine sample, a saliva sample, a lymph sample, a pleural fluid sample, a mucosa sample, and a combination thereof.
